(19) United States
(12) Patent Application Publication
(10) Pub. No.: US 2008/0300217 A1
(43) Pub. Date: Dec. 4, 2008

(54) Combination Therapy with Fumaric Acid Esters for the Treatment of Autoimmune and/or Inflammatory Disorders

(75) Inventor: Henrik Nilsson, Copenhagen (DK)

Correspondence Address: DARBY & DARBY P.C., P.O. BOX 770, Church Street Station, New York, NY, 10008-0770, US

(73) Assignee: ADITECH PHARMA AB, Malmo (SE)

(21) Appl. No.: 12/089,004

(22) PCT Filed: Oct. 9, 2006
(86) PCT No.: PCT/DK06/00563

§ 371 Date: Apr. 2, 2008

Related U.S. Application Data
(60) Provisional application No. 60/744,458, filed on Apr. 7, 2006.

(30) Foreign Application Priority Data
Oct. 7, 2005 (DK) PCT/DK2005/000648
Apr. 7, 2006 (DK) PA 2006 00511

(51) Int. Cl. A61K 31/736; A61K 31/225; A61K 31/403; A61K 31/60; A61K 31/5415; A61K 31/192; A61P 17/06; A61K 31/352; A61K 31/167; A61P 29/00; A61P 37/02; A61K 31/196

(52) U.S. Cl. 514/54; 514/547; 514/411; 514/163; 514/226.5; 514/569; 514/629; 514/567; 514/456

(57) ABSTRACT

The present invention relates to compositions, kits and methods for administration of a first component of fumaric acid ester(s), or a pharmaceutically acceptable salt thereof, and a second component which is a substance that reduces or eliminates flushing.

### FIELD OF THE INVENTION

The present invention relates to compositions, kits and methods for administration of a first component of fumaric acid ester(s), or a pharmaceutically acceptable salt thereof, and a second component which is a substance that reduces or eliminates flushing.

### BACKGROUND

Fumaric acid esters, i.e. dimethylfumarate in combination with ethylhydrogenfumarat have been used in the treatment of psoriasis for many years. The combination is marketed under the tradename Fumaderm®. It is in the form of tablets intended for oral use and it is available in two different dosage strengths (Fumaderm® initial and Fumaderm®):

| | Fumaderm ® Initial | Fumaderm ® |
|---|---|---|
| Dimethylfumarate | 30 mg | 120 mg |
| Ethylhydrogenfumarate, | 67 mg | 87 mg |
| calcium salt | | |
| Ethylhydrogenfumarate, | 5 mg | 5 mg |
| Magnesium salt | | |
| Etylhydrogenfumarate, | 3 mg | 3 mg |
| Zinc salt | | |

The two strengths are intended to be applied in an individually based dose regimen starting with Fumaderm® initial in an escalating dose, and then after e.g. three weeks of treatment switching to Fumaderm®. Both Fumaderm® initial and Fumaderm® are enteric coated tablets.

Another marketed composition is Fumaraat 120® containing 120 mg of dimethylfumarate and 95 mg of calcium monoethylfumarate (TioFarma, Oud-Beijerland, Netherlands). In a recent publication (Litjens et al. Br. J. Clin. Pharmacol. 2004, vol. 58:4, pp. 429-432), the pharmacokinetic profile of Fumaraat 120® is described in healthy subjects.

U.S. Pat. No. 6,277,882 and U.S. Pat. No. 6,355,676 disclose respectively the use of alkyl hydrogen fumarates and the use of certain fumaric acid mono alkyl ester salts for preparing micro tablets for treating psoriasis, psoriatic arthritis, neurodermatitis and enteritis regionalis Crohn. U.S. Pat. No. 6,509,376 disclose the use of certain dialkyl fumarates for the preparation of pharmaceutical preparations for use in transplantation medicine or the therapy of autoimmune diseases in the form of micro tablets or pellets. U.S. Pat. No. 4,959,389 disclose compositions containing different salts of fumaric acid monoalkyl ester alone or in combination with dialkyl fumarate. GB 1,153,927 relates to medical compositions comprising dimethylmaleic anhydride and/or dimethylmaleic acid and/or a dimethylfumaric acid compounds. The Case report “Treatment of disseminated granuloma annulare with fumaric acid esters” from BMC Dermatology, vol. 2, no. 5, 2002, relates to treatment with fumaric acid esters.

However, therapy with fumarates like e.g. Fumaderm® frequently gives rise to some side effects such as flushing.

It is known from Friedrich et al: “Addition of Pentoxifylline Could Reduce the Side Effects of Fumaric Acid Esters in the Treatment of Psoriasis”, Acta Derm Venereol 2001; 81:429-451 that the addition of pentoxifylline to the standard Fumaderm treatment reduced the flush symptoms in patients.

One object of the present invention is to eliminate or reduce substantial flushing (frequency and/or severity) as a side effect during the treatment of an autoimmune and/or inflammatory disorder such as psoriasis and related conditions using fumaric acid esters.

Another object of the present invention is to provide a combination therapy for an autoimmune and/or inflammatory disorder such as psoriasis and related conditions that minimizes side effects generally.

Another object of the present invention is to provide a combination therapy for an autoimmune and/or inflammatory disorder such as psoriasis and related conditions that enables a more effective treatment with higher doses of fumaric acid esters.

Yet another object is to provide a pharmaceutical composition for oral use, such as a fixed combination.

These and other objects will be apparent from the description provided herein.

### SUMMARY OF THE INVENTION

The present invention provides a method of treating an autoimmune and/or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and in a therapeutically effective amount a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt.

The present invention also provides a method of treating an autoimmune and/or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and in a therapeutically effective amount a second component which is a substance that reduces or eliminates flushing.

The present invention also provides the use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt;
   for the manufacture of a medicament for treating an autoimmune and/or inflammatory disorder.

The present invention also provides the use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and a second component which is a substance that reduces or eliminates flushing for the manufacture of a medicament for treating an autoimmune and/or inflammatory disorder.

The present invention also provides a pharmaceutical composition which comprises a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing.

### DETAILED DESCRIPTION

As used herein the term “autoimmune and/or inflammatory disorder” is meant to encompass any disorder characterized by the host's immune system reacting against the host's own antigens and/or acute inflammation and/or chronic inflammation and comprises the following specific disorders: psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica and granuloma annulare.

The term “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician.

In the present context the term “flushing” describes episodic attacks of redness of the skin together with a sensation of warmth or burning of the face, neck, and less frequently the upper trunk and abdomen. It is the transient nature of the attacks that distinguishes flushing from the persistent erythema of photosensitivity or acute contact reactions. Repeated flushing over a prolonged period of time can lead to telangiectasia and occasionally to classical rosacea of the face (Greaves M W. Flushing and flushing syndromes, rosacea and perioral dermatitis. In: Champion R H, et al, eds. Rook/Wilkinson/Ebling textbook of dermatology, 6^{th} ed., vol. 3. Oxford, UK: Blackwell Scientific, 1998: 2099-2104). As used herein the term “a substance that reduces or eliminates flushing” refers to a substance that will reduce or eliminate the flushing (in terms of frequency and/or severity of the episodes) as a side effect during the treatment of humans for an autoimmune and/or inflammatory disorder with fumaric acid ester(s). The flushing effect of fumaric acid ester(s) usually becomes less frequent and less severe as the patient develops tolerance to the flushing side effect at therapeutic doses of the drug, but the flushing effect still may occur to some extent. Thus, “a substance that reduces or eliminates flushing” refers to the ability of a substance to reduce the severity of flushing when it occurs, or result in fewer flushing events than would otherwise occur. As used in this context the term “therapeutically effective amount” means an amount sufficient to reduce the severity of flushing when it occurs, or result in fewer flushing events than would otherwise occur. An amount adequate to accomplish this is defined as a “therapeutically effective amount”.

In the present context, a reduction of flushing is intended to denote a decrease in severity and/or incidence/frequency among a given treated patient population, compared to the flushing observed after administration of the first component in combination with a placebo and can be measured e.g as described by O'toole et al. Cancer 2000, 88(4): p. 770-776. A reduction in flushing according to this definition could thus be construed as a substantial reduction in incidence or severity of flushing. In one aspect of the invention, the incidence of flushing is reduced by at least about a third, in another aspect of the invention the incidence is reduced by half, and in a further aspect of the invention, the flushing incidence is reduced by about two thirds or more. Likewise, the severity is in one aspect of the invention reduced by at least about a third, in another aspect of the invention by at least half, and in a further aspect of the invention by at least about two thirds. Clearly a one hundred percent reduction in flushing incidence and severity is most preferable, but is not required. The reduction of flushing, as described above, can be monitored in a clinical trial setting, such as comparing the administration of the first component with the second component of the present invention compared with treatment of the first component in combination with a placebo. In case of a placebo controlled trial, the incidence and severity, defined as mild, moderate or severe, of flushing in the patients receiving the adjunctive treatment according to the invention compared to the placebo group, can be compared. Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the psoriasis area severity index (PASI). Typically, patients within a certain range of PASI are included, such as between 10 and 40, or such as between 12 and 30, or such as between 15 and 25 or ≧10 or ≧12 or ≧16. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group are sufficient in most cases, but more preferably about 30 to 50 patients are included in each arm of the study. Total study duration can be as short as one day to one week, but more preferably the study will run for 8 weeks to 12 weeks or up to 16 weeks. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

In one aspect, the invention relates to a method of treating an autoimmune and/or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and in a therapeutically effective amount a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt.

In another aspect, the invention relates to a method of treating an autoimmune and/or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and in a therapeutically effective amount a second component which is a substance that reduces or eliminates flushing.

In another aspect, the invention relates to a method of treating an autoimmune and/or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and in a therapeutically effective amount a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals,
d. a mineral salt, and
e. a TNF alpha inhibitor.

In another aspect, the invention relates to use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt;
   for the manufacture of a medicament for treating an autoimmune and/or inflammatory disorder.

In another aspect, the invention relates to the use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and a second component which is a substance that reduces or eliminates flushing, such as a prostaglandin modulator, a dietary fiber, a composition comprising inhibitors of mast cell activation and secretion of inflammatory biochemicals, a mineral salt, or a TNF alpha inhibitor, for the manufacture of a medicament for treating an autoimmune and/or inflammatory disorder.

In another aspect, the invention relates to a pharmaceutical composition which comprises a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing, such as a prostaglandin modulator, a dietary fiber, a composition comprising inhibitors of mast cell activation and secretion of inflammatory biochemicals, a mineral salt, or a TNF alpha inhibitor.

In one aspect of the invention, the second component is a TNF alpha inhibitor.

As used herein the term “TNF alpha inhibitor” comprises infliximab, adalimumab, CDP870, CDP571, etanercept, pentoxifylline, onercept, TNF-alpha VAC, humicade, certolizumab pegol, lenalidomide, ITF-2357, lenercept, melanine analogues (LSB), PASSTNF-alpha, POL-647, TBC-16011, thalidomide (Andrulis), thamidomide (Celgene), Pharmaprojects no. 6181, antisense compounds (Elan), trochodimerol, xyloadenosine, M-PGA, tgAAC94, ISIS-104838, delmitide acetate, AGIX-4207, CLX-1100, TNF-alpha antagonists (Dynavax), Genz-29155, CDC-394, NPI-1302a-3, RDP-59, VT-346, ENMD-0995, ENMD-0997, PLR-14, UR-1505, TNF-alpha inhibitors (Morphochem), Genz-34940, LMP-420, LMP-160, ethylpuruvate, STA-6292, AME-527, golumumab, REN-1654, Dom-0200, ISIS-104838, ABX/0401, CR-1, WP9QY, S5, ABX/0402, CC-11006, IMiDs (class III, Celgene), CC-10004, CC11050, PG-8395, NBE-P2, BKT-104, CYT007-TNFQb, ESBA-105, CC-10015, Dom-0215, Dom-0100, siRNA RA therapy, AN-0128, PMI-006, PMI-005 and Y-40138.

In a further aspect, the invention relates to oral TNF alpha inhibitors.

In another aspect, the invention relates to an oral TNF alpha inhibitor selected from the group consisting of lenalidomide, ITF-2357, POL-647, thalidomide (Andrulis), thalidomide (Celgene), Pharmaprojects No. 6181, antisense compounds (Elan), antisense compounds (Isis), delmitide acetate, AGIX-4207, TNF-alpha antagonists (Dynavax), Genz-29155, CDC-394, NPI-1302a-3, ENMD-0995, ENMD-0997, PLR-14, UR-1505, TNF-alpha inhibitors (Morphochem), LMP-420, LMP-160, STA-6292, ISIS-104838, ABX/0402, CC-11006, IMiDs (class III, Celgene), CC-10004, CC-11050, PG-8395 and BKT-104.

In one aspect of the invention, the second component is a prostaglandin modulator.

As used herein the term “prostaglandin modulator” comprises NSAIDs, corticosteroids and prostaglandin inhibitors.

In one aspect of the invention, the second component is a prostaglandin modulator selected from the group consisting of NSAIDs, corticosteroids and prostaglandin inhibitors

Examples of a corticosteroid comprise betamethasone, desoximethasone, fluocinolone, momethasone, hydrocortisone aceponate, fluticasone, clobethasol, clobethasone, hydrocortisone butyrate, desonide, triamcinolone or hydrocortisone.

In one aspect of the invention, the second component is a prostaglandin inhibitor.

As used herein the term “prostaglandin inhibitor” comprises prostaglandin D2 inhibitors.

In one aspect of the invention, the second component is a prostaglandin D2 inhibitor.

As used herein the term “prostaglandin D2 inhibitor” includes but are not limited to ZK-110841, ZK-118182, S-5751, ONO-4127Na, L-888839 and SAR-389644.

Prostaglandin D2 interacts with different subtypes of receptors. One prostaglandin D2 receptor is referred to as “DP” and another prostaglandin D2 receptor is known as “CRTH2”.

In one aspect of the invention, the second component is a DP receptor antagonist.

One aspect of the invention relates to the use of a DP receptor antagonist that selectively modulates the DP receptor without substantially modulating the CRTH2 receptor. Thus, the DP receptor antagonist has an affinity at the DP receptor (i.e., Kᵢ) that is at least about 10 times higher (a numerically lower Kᵢ value) than the affinity at the CRTH2 receptor. Any compound that selectively interacts with DP according to these guidelines is deemed “DP selective”.

As used herein the term “DP receptor antagonist” comprises the compounds disclosed in WO2004/103370 which is incorporated herein by reference, especially the compounds described as compound A through AJ and the pharmaceutically acceptable salts and solvates thereof.

In one aspect of the invention, the second component is the compounds described as compound A through AJ and the pharmaceutically acceptable salts and solvates thereof disclosed in WO2004/103370, page 4-6, such as compound D

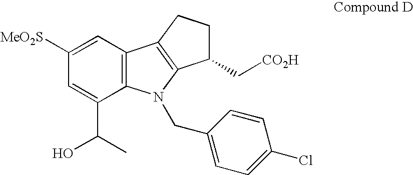

In another aspect of the invention, the second component is MK-0524.

In another aspect of the invention, the second component is a NSAID.

As used herein the term “NSAID” is defined to mean “non-steroidal anti-inflammatory drug”. The mechanism of the NSAID's action is believed to be its acetylation and deactivation of cyclooxygenase (of which there are three kinds presently known, COX-1, COX-2 and COX-3, and different NSAIDs may display different degrees of deactivation/inhibition of the three cyclooxygenases), thereby reducing the amount of active enzyme and its prostaglandin (PG) D2 product to a level which does not produce significant flushing. In one aspect of the invention, the NSAID is more selective towards COX-2 than COX-1 and COX-3. Such a compound is termed a COX-2 inhibitor In the present context. The NSAID can be a sustained release form, immediate release form or combination thereof, and it can optionally include a delayed release form of NSAID. Examples of NSAIDs comprises salicylates such as aspirin and salicylate salts; propionic acids such as ibuprofen, fenoprofen, suprofen, benoxaprofen, flurbiprofen, ketoprofen, carprofen, naproxen, and sodium naproxen; indoleacetic acid derivatives such as indomethacin, sulindac, and etodolac; benzeneacetic acids such as aclofenac, diclofenac, and fenclofenac; pyrroleacetic acids such as tolmectin and zomepirac; anthranilic acids such as meclofenamate and mefenamic acid; pyrazoles such as oxyphenbutazone and phenylbutazone; oxicams such as piroxicam; nabumetone; paracetamol (acetaminophen); meloxicam; and apazone. The NSAID composition can also include a pharmaceutically acceptable, nontoxic carboxylic acid compound in an amount which is sufficient to increase the effectiveness of the method for a NSAID resistant subject. Examples of NSAID compositions are disclosed in WO 96/32942 which is incorporated by reference.

In one aspect of the invention, the second component is a NSAID selected from the group consisting of meloxicam, piroxicam, naproxen, acetaminophen (paracetamol), ibuprofen, dexibuprofen, diclofenac, and salicylic acid.

In another aspect of the invention, the second component is a NSAID selected from the group consisting of meloxicam, piroxicam, naproxen, ibuprofen, dexibuprofen, diclofenac, and salicylic acid.

In yet another aspect of the invention, the second component is a NSAID selected from the group consisting of meloxicam, piroxicam, naproxen, ibuprofen, dexibuprofen and diclofenac.

Examples of suitable COX-2 inhibitors are rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6-dimethyl-6H-dibenzo[b,d]pyran carboxylic acid (CT-3), 2(5H)-Furanone, 5,5-dimethyl (1-methylethoxy) [4(methylsulfonyl)phenyl]-(DFP); Carprofen (RIMADYL), (Acetyloxy)-benzoic acid, 3-[(nitrooxy)methyl]phenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenol (S-2474), 5(R)-Thio sulfonamide-3(2H)-benzofuranone (SVT-2016) and N-[3-(Formyl-amino) oxo phenoxy-4H benzopyran yl]methanesulfonamide (“T-614”); or a pharmaceutically acceptable salt thereof.

In one aspect of the invention, the second component is a dietary fiber.

As used herein the term “dietary fibers” is defined as “remnants of plant cells resistant to hydrolysis by the alimentary enzymes of man, the group of substances that remain in the ileum but are partly hydrolyzed by bacteria in the colon”, according to JAMA 262, pp. 542-546 No. 4 (Jul. 28, 1989) Council Report entitled “Dietary Fiber and Health”, page 542. This article, moreover, gives considerable information as to what constitutes a “dietary fiber” and is accordingly incorporated herein by reference. Gel-forming dietary fibers include mucillages, plant gums, pectins or pectic substances, and lignin, all of which are endogenous compounds of plant materials which are resistant to digestion by enzymes in the monogastric stomach or small intestine. Chemically, nearly all of these plant materials are carbohydrates composed of repeating monosaccharide (sugar) units. Disaccharides have two sugar units, oligosaccharides three to twelve, and polysaccharides may contain a million or more. The water-soluble fractions of these substances form gels in the stomach and intestinal tract and lower serum cholesterol.

Gums and mucillages have no common structure but are polysaccharides containing several sugars with alternating monomer structures, and may or may not contain uronic acids. There are many gums found in plants and cereal grains. Guar and locust bean gums are galactomannans, whereas gum arabic is an acidic polymer of galactose and rhammose. Oat and barley contain gums, but are not practical for use in the present application because of the low percentage of active gums per weight volume. Large amounts of oat bran (e.g., 100 grams per day) are also required to lower serum cholesterol. Most of the gums in the context of the present application are effective at much lower dosages. Suitable gums include, inter alia, besides guar gum, the following: locust bean gum, acacia gum, gum arabic, xanthan gum, carrageenan gum, karaya gum, tragacanth gum, and ghatti gum.

Pectin substances or pectins are mixtures of polysaccharides of partially methylated and 1,4-D galacturonic acid units with side chains containing arabinose, galactose, xylose, and rhammose. They are contained in many fruits and vegetables as well as other plants.

Other suitable gel-forming dietary fibers include psyllium husks, algal polysaccharides, glucomannan, and agar, to name a few. Lignin is a non-carbohydrate polymer of aromatic plant alcohols comprising oxygenated phenylpropane units. As a plant matures, more lignin is produced, which acts as a sort of cement as it hardens and holds together other plant cell wall constituents. Lignin passes through the digestive tract with very little change.

As already mentioned, a review of dietary fiber which mentions these substances is contained in the following reference: Dietary Fiber and Health, JAMA 262: 542-546 (1989), from the Council on Scientific Affairs, American Medical Association.

U.S. Pat. No. 5,023,245 describes different types and formulations of dietary fibers, and is incorporated herein by reference.

The dietary fibers may take the form of the usual tablets, capsules, suspensions, dispersions, elixirs, syrups, or the like, whether administered singly or in combination, and may moreover be provided in the usual form for dietary supplements involving inclusion of a fibrous material, such as in capsules, drink mixes, breakfast foods, or the like, especially when metallic salts assisting with the internal dispersion of the guar gum or other gel-forming dietary fiber are included and/or when acids, and especially organic acids such as citric, ascorbic, malic, and tartaric are included not only to delay gelation of the guar gum or other gel-forming dietary fiber when the active ingredients are presented in the form of a drink mix but also to add a pleasant palatable flavor thereto.

One aspect of the invention, relates to the use of a guar gum that reduces or eliminates flushing as the second component. Examples of such guar gum are disclosed in U.S. Pat. No. 5,023,245 incorporated herein by reference.

One aspect of the invention, relates to the use as a second component of a mineral and/or metal salt that is soluble in the gastrointestinal fluids, such mineral salt may be provided as a buffer or to enhance dispersability of a gel forming dietary fiber. Examples of such mineral salts are disclosed in U.S. Pat. No. 5,023,245. One specific example of a mineral salt which may be soluble in the gastrointestinal fluids, is a magnesium salt, which, in addition to the benefits described above, in itself may reduce flushing and/or itching. In one aspect of the invention, the second component is calcium carbonate, magnesium carbonate or potassium carbonate.

One aspect of the invention, relates to the use as a second component of an inorganic magnesium salt that reduces or eliminates flushing. Examples of such inorganic magnesium salts are disclosed in U.S. Pat. No. 4,911,917 incorporated herein by reference. In one aspect of the invention, the mineral is magnesium oxide or magnesium carbonate.

In one aspect of the invention, the second component is a composition comprising inhibitors of mast cell activation and secretion of inflammatory biochemicals.

As used herein the term “a composition comprising inhibitors of mast cell activation and secretion of inflammatory biochemicals” comprises a combination of a sulfated proteoglycan, with or without a unique unrefined olive kernel extract, with one or more of a sulfated D-hexoseamine, a flavone or isoflavone, CRH antagonists, histamine-I receptor antagonists, histamine-3 receptor agonists, polyamines, rutin and caffeine as described in US 2005/0220909 incorporated herein by reference. Although not bound by any particular mechanism of action, it is contemplated that the proteoglycan inhibits the activation and degranulation of the relevant mast cells, while the flavone inhibits the secretion of inflammatory biomolecules from these mast cells. “Activation” and “degranulation” of mast cells are defined herein as is standard and well known in this art, that is, to mean synthesis and secretion from the activated mast cell of any type of molecule(s) that alone or in combination triggers inflammatory processes. Furthermore, the term “a composition comprising inhibitors of mast cell activation and secretion of inflammatory biochemicals” also comprises mast cell stabilizers, such as sodium chromoglycate.

The dosages of the drugs used in the present invention must, in the final analysis, be set by the physician in charge of the case, using knowledge of the drugs, the properties of the drugs in combination as determined in clinical trials, and the characteristics of the patient, including diseases other than that for which the physician is treating the patient.

General outlines of the dosages, and some preferred dosages will be provided here. Dosage for some of the drugs will be given in order to create a guideline for any of the desired combinations.

Dietary fiber/guar gum: from about 250 to about 6000 mg, such as from about 400 to about 500 mg per dose. In one aspect of the invention, the dietary fiber such as guar gum is administered once to three times daily.

Physiologically acceptable magnesium salt, such as magnesium carbonate: from about 50 to about 1500 mg, such as from about 80 to about 500 mg, such as from about 80 to about 100 mg per dose. In one aspect of the invention, the magnesium salt such as magnesium carbonate is administered once to three times daily.

Paracetamol: from about 500 to about 4000 mg daily, such as from about 1000 to about 4000 mg daily, such as from about 1000 to about 3000 mg daily, such as from about 1000 to about 2000 mg daily. Alternatively, from about 500 to about 1000 mg per dose.

Salicylic acid (ASA): from about 1 to about 3000 mg daily, such as from about 75 to about 320 mg daily, such as 75, 81, 160 mg or 300 mg once or twice daily (or per dose).

Examples of dosages of selective COX-2 inhibitors is: rofecoxib (Vioxx) dosed in the range corresponding to 10-50 mg/day, valdecoxib (Bextra) dosed in the range corresponding to 5-20 mg/day, celecoxib (Celebrex) dosed in the range corresponding to 100-500 mg/day, etoricoxib (Arcoxia) dosed in the range corresponding to 25-150 mg/day, lumiracoxib (Prexige) dosed in the range corresponding to 100-500 mg/day, parecoxib (Dynastat) dosed in the range corresponding to 20-200 mg/day, deracoxib (Deram) dosed in the range corresponding to 10-200 mg/day.

Meloxicam: from about 5 to about 20 mg/day,
Piroxicam: from about 10 to about 30 mg/day.
Naproxen: from about 500 to about 1500 mg/day.
Dexibuprofen: from about 500 to about 1600 mg/day.
Ibuprofen: from about 1000 to about 3200 mg/day.
Salsalate: from about 1000 to about 3000 mg/day.
CINOD AZD3582: from about 200 to about 2000 mg/day.
Diclofenac: from about 50 to about 150 mg daily, or from about 50 to about 75 mg per dose.

The first component comprises in one aspect of the invention one or more fumaric acid ester(s). In one aspect of the invention the fumaric acid ester is preferably selected from the group consisting of dimethylfumarate, diethylfumarate, dipropylfumarate, dibutylfumarate, dipentylfumarate, methyl-ethylfumarate, methyl-propylfumarate, methyl-butylfumarate, methyl-pentylfumarate, monomethylfumarate, monoethylfumarate, monopropylfumarate, monobutylfumarate and monopentylfumarate, including pharmaceutically acceptable salts thereof.

In a specific embodiment of the invention, the fumaric acid ester is a mono-(C₁-C₅)alkylester of fumaric acid that is present in the form of a pharmaceutically acceptable salt. Suitable salts are e.g. metal salts such as a salt selected from alkali metal salts and alkaline earth metal salts including sodium, potassium, calcium, magnesium or zinc salt.

The term (C₁-C₅)alkyl refers to a branched or un-branched alkyl group having from one to five carbon atoms inclusive, such as methyl, ethyl, 1-propyl, 2-propyl, 1-butyl, 2-butyl, 2-methyl-2-propyl, 2-methyl-1-propyl and pentyl.

In another embodiment, the first component comprises dimethylfumarate as the active substance.

In a further embodiment, the first component comprises monomethylfumarate as the active substance optionally in the form of a pharmaceutically acceptable salt like e.g. its sodium, potassium, calcium, magnesium and/or zinc salt.

In another embodiment, the first component consists essentially of dimethylfumarate as the active substance.

In another embodiment, the first component consists of dimethylfumarate as the active substance.

In a further embodiment, the first component consists essentially of monomethylfumarate as the active substance optionally in the form of a pharmaceutically acceptable salt like e.g. its sodium, potassium, calcium, magnesium and/or zinc salt.

In a further embodiment, the first component consists of monomethylfumarate as the first active substance optionally in the form of a pharmaceutically acceptable salt like e.g. its sodium, potassium, calcium, magnesium and/or zinc salt.

In a further embodiment, the first component comprises dimethylfumarate and monomethylfumarate (optionally in the form of a pharmaceutically acceptable salt like e.g. its sodium, potassium, calcium, magnesium and/or zinc salt) as the active substance, in a weight ratio between about 1:10 and about 10:1.

In a further embodiment, the first component consists essentially of dimethylfumarate and monomethylfumarate (optionally in the form of a pharmaceutically acceptable salt like e.g. its sodium, potassium, calcium, magnesium and/or zinc salt) as the active substance, in a weight ratio between about 1:10 and about 10:1.

In a further embodiment, the first component consists of dimethylfumarate and monomethylfumarate (optionally in the form of a pharmaceutically acceptable salt like e.g. its sodium, potassium, calcium, magnesium and/or zinc salt) as the active substance, in a weight ratio between about 1:10 and about 10:1.

In one aspect of the invention, the first component is contained in Fumaderm® or Fumaraat® or Panaclar® (BG-12) or as described in U.S. Pat. No. 6,277,882, U.S. Pat. No. 6,355,676 or U.S. Pat. No. 6,509,376.

In one aspect of the invention, the first component is provided in the form of Fumaderm®.

In another aspect of the invention, the first component is provided in the form of Panaclar® (BG-12).

In one aspect of the invention, the first component is provided in the form of Fumaraat®.

In one aspect of the invention, the first component is formulated in a composition that enables a prolonged and/or a slow release of a fumaric acid ester as defined above. Examples of such compositions are for example described in PCT/DK2005/000648 which is hereby incorporated by reference.

In the present context, a controlled release fumaric acid ester composition is a composition that is designed to release the fumaric acid ester in a prolonged, slow and/or delayed manner compared to the release of the commercially available product Fumaderm®, when tested under comparable conditions (e.g. for in vivo studies: dose equivalents, with or without standardized meal etc., or for in vitro studies: dose equivalents, dissolution test apparatus and working conditions including e.g. composition, volume and temperature of dissolution medium employed, rotation speed etc.).

The release in vivo may be tested by measuring the plasma concentration at predetermined time periods and thereby obtaining a plasma concentration versus time profile for the fumaric acid ester in question or, if relevant, a metabolite thereof. (E.g. in the case of dimethylfumarate, the active substance is envisaged to be methylhydrogenfumarate, i.e. the monomethyl ester of fumaric acid). Furthermore, it is contemplated that metabolism already takes place within the gastrointestinal tract or during passage of the gastrointestinal mucosa, or upon first passage through the hepatic circulation. Accordingly, when dimethylfumarate is administered, the relevant component to search for in the plasma may be the monomethyl ester and not the dimethylester of fumaric acid.

Other tests may also be used to determine or to give a measure of the release of the active substance in vivo. Thus, animals (e.g. mice, rats, dogs etc.) may be used as a model. The animals receive the compositions under investigation and after specified periods of time, the animals are sacrificed and the content of the active ingredient (or metabolite thereof, if relevant) is determined in plasma or specific organs or extracted from the intestinal contents.

Another test involves the use of a specific segment of an animal intestine. The segment is placed in a suitable dissolution apparatus containing two compartments (a donor and a receiver) separated by the segment, and the composition under investigation is placed in a suitable medium in one compartment (the donor compartment). The composition will release the active substance that subsequently is transported across the intestinal segment.

Accordingly, at suitable time intervals, the concentration of the active substance (or, if relevant, the metabolite) is measured in the receiver compartment.

A person skilled in the art will be able to adapt the above-mentioned method to the specific composition.

With respect to in vitro methods, well-established methods are available, especially methods described by official monographs like e.g. United States Pharmacopeia (USP) or the European Pharmacopoeia. A person skilled in the art will know which method to choose and how to select the specific conditions to carry out the in vitro test. For instance, the USP prescribes in vitro tests be carried out at 37+/−1.0 such as 37+/−0.5 degrees Celsius/Centigrade. A suitable dissolution test is, for example for capsules, wherein the dissolution profile is determined as described in the United States Pharmacopoeia at 37° C. using a rotating basket at 100 rpm employing 0.1 N hydrochloric acid as dissolution medium during the first 2 hours of the test and then followed by 0.05 M phosphate buffer pH 6.5 as dissolution medium for the remaining test period, and, for example as described for tablets wherein the dissolution profile is determined as described in the United States Pharmacopoeia at 37° C. using a paddle dissolution apparatus at 100 rpm employing 0.1 N hydrochloric acid as dissolution medium during the first 2 hours of the test and then followed by 0.05 M phosphate buffer pH 6.5 as dissolution medium for the remaining test period.

As mentioned above, the in vivo release of the first component is in one aspect of the invention prolonged, slow and/or delayed compared with the commercially available Fumaderm® composition.

With regard to the first component, the term “prolonged” is intended to indicate that the active substance is released during a longer time period than Fumaderm® such as at least during a time period that is at least 1.2 times, such as, e.g., at least 1.5 times, at least 2 times, at least 3 times, at least 4 times or at least 5 times greater than that of Fumaderm®. Thus, if e.g. 100% of dimethylfumarate is released from Fumaderm® tablets 3 hours after the start of a suitable test, then 100% of dimethylfumarate in a composition according to the invention is released at least 3.6 hours after the start of a suitable test.

With regard to the first active substance the term “delayed” is intended to indicate that the release of the first active substance starts at a later point in time compared with that of Fumaderm® (such as at 30 min or more later such as, e.g., 45 min or more later, 1 hour or more later or 1.5 hours or more later, alternatively, that the initial release during the first 2 hours is much less compared with that of Fumaderm® (i.e. less than 80% w/w such as, e.g., less than 70% w/w, less than 60% w/w or less than 50% of that of Fumaderm®).

An example of a particular useful composition comprising the first component is a controlled release fumaric acid ester composition designed to be administered two or more times daily described in PCT/DK2005/000648 which is hereby incorporated by reference.

Accordingly, the present invention relates in a further aspect to the use of a first component provided in a controlled release pharmaceutical composition for oral use comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, wherein the release of the fumaric acid ester—when subjected to an in vitro dissolution test employing 0.1 N hydrochloric acid as dissolution medium during the first 2 hours of the test and then 0.05 M phosphate buffer pH 6.5 as dissolution medium—is as follows:

within the first 3 hours after start of the test at the most about 70% w/w of the total amount of the fumaric acid ester contained in the composition is released, and/or
within the first 4 hours after start of the test at the most about 92% w/w of the total amount of the fumaric acid ester is released, and/or
within the first 5 hours after start of the test at the most about 94% w/w of the total amount of the fumaric acid ester is released, and/or
within the first 6 hours after start of the test at the most about 95% w/w of the total amount of the fumaric acid ester contained in the composition is released, and/or
within the first 7 hours after start of the test at the most about 98% w/w of the total amount of the fumaric acid ester contained in the composition is released, and/or
within the first 9 hours after start of the test at the most about 99% w/w of the total amount of the fumaric acid ester contained in the composition is released and/or
within the first 12 hours after start of the test at the most about 99% w/w of the total amount of the fumaric acid ester contained in the composition is released.

In the following is given a description of specific embodiments, wherein the fumaric acid ester is released depending on pH and wherein the release pattern is suitable for compositions that are administered two or more times daily. Examples of suitable formulation principles are e.g. compositions provided with an enteric coating or hydrogels of a type described by Zentner et al (U.S. Pat. No. 6,537,584) and Bae (U.S. Pat. No. 5,484,610), which hereby are incorporated by reference. Further examples of suitable formulation principles are e.g. compositions provided with a diffusion coating such as a controlled release diffusion coating, matrix particulates or matrix tablets, hydrogels, pulsed dose drug delivery systems, co-formulation with vitamin E concentrate or ethanol, TPGS, corn oil and wax etc., including any of the formulation principles mentioned above, optionally with an enteric coating.

In another aspect, the invention relates to the use of a first component provided in a controlled release pharmaceutical composition for oral use comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to a in vitro profile of dissolution when measured according to USP in 0.1 N hydrochloric acid during the first 2 hours and then 0.05 M phosphate buffer at a pH 6.5 or 6.8,

wherein at the most 5% w/w of the total amount of the fumaric acid ester contained in the composition is released within the first 2 hours after start of the test, and/or
wherein from about 20% to about 75% w/w of the total amount of the fumaric acid ester contained in the composition is released within the first 3 hours after start of the test, and/or
wherein from about 50% to about 90% w/w of the total amount of the fumaric acid ester contained in the composition is released within the first 4 hours after start of the test, and/or
wherein from about 60% to about 90% w/w of the total amount of the fumaric acid ester contained in the composition is released within the first 5 hours after start of the test, and/or
wherein from about 70% to about 95% w/w of the total amount of the fumaric acid ester contained in the composition is released within the first 6 hours after start of the test, and/or
wherein from about 75% to about 97% w/w of the total amount of the fumaric acid ester contained in the composition is released within the first 7 hours after start of the test, and/or
wherein at least 85% w/w of the total amount of the fumaric acid ester contained in the composition is released within the first 8 hours after start of the test.

In a further aspect, the invention relates to the use of a first component provided in a controlled release pharmaceutical composition for oral use comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to a in vitro profile of dissolution when measured according to USP in 0.1 N hydrochloric acid during the first 2 hours and then 0.05 M phosphate buffer at a pH 6.5 or 6.8, wherein at the most 5% w/w of the total amount of the fumaric acid ester contained in the composition is released within the first 2 hours after start of the test, wherein from about 20% to about 75% w/w of the total amount of the fumaric acid ester contained in the composition is released within the first 3 hours after start of the test, wherein from about 50% to about 90% w/w of the total amount of the fumaric acid ester contained in the composition is released within the first 4 hours after start of the test, wherein from about 60% to about 90% w/w of the total amount of the fumaric acid ester contained in the composition is released within the first 5 hours after start of the test, wherein from about 70% to about 95% w/w of the total amount of the fumaric acid ester contained in the composition is released within the first 6 hours after start of the test, wherein from about 75% to about 97% w/w of the total amount of the fumaric acid ester contained in the composition is released within the first 7 hours after start of the test, and wherein at least 85% w/w of the total amount of the fumaric acid ester contained in the composition is released within the first 8 hours after start of the test.

Typically, as described above, the controlled release compositions comprising the first component are designed to deliver the first component and/or the active substance thereof (i.e. the monoalkylester of fumaric acid, which in turn is metabolised to fumaric acid and, which subsequently is subjected to a rapid elimination process) in a prolonged manner. Apart from the characteristic in vitro release patterns described herein, such a prolonged release is reflected in the pharmacokinetic parameters obtained after a clinical study as well. Accordingly, it is contemplated in one aspect of the invention that the Cₘₐₓ of the monoalkylester of fumaric acid (which appears in the plasma upon hydrolysis or metabolism of the dialkylester administered) is of the same order of magnitude as previously described in the literature provided that a similar or equivalent dose is administered (i.e. Cₘₐₓ of monomethylfumarate in a range of from about 0.4 to about 3.2 mg/l, such as of from about 0.4 to about 2.0 mg/l, corresponding to an oral dose of 120 to 240 mg dimethylfumarate). However, in order to avoid many frequent daily administrations (2-4 tablets 1-3 times daily) it is an aim to prolong the time period where the concentration is within the therapeutic window. Accordingly, it is contemplated that W₅₀ (i.e. the time period in which the plasma concentration is 50% of Cₘₐₓ or more) is prolonged compared to the marketed treatment with at least 100% such as, e.g. at least 20%, at least 30%, at least 40% or at least 50%. A suitable W₅₀ is believed to be at least 2 hours such as in a range of from about 2 to about 15 hours or from about 2.5 to about 10 hours or from about 3 to about 8 hours.

Furthermore, it is contemplated that a controlled release composition may lead to a reduced interindividual and/or intraindividual variation in the plasma profile and to a reduced dependency on whether the composition is taken together with or without food (a reduced variation of the plasma concentration profile of monomethylfumarate when the pharmaceutical composition is administered with or without concomitant food intake). Therefore, the controlled release composition comprising the first component may lead to a reduced frequency of dosing and/or a reduced average total daily dose, and/or an increased efficacy at the same total daily dose of the active substance compared to Fumaderm®.

Different kinetic models, such as zero-order (1), first-order (2), square-root (Higuchi's equation) (3) can be applied to the interpretation of the drug release kinetic.

Mₜ=M₀+k₀*t 1

ln Mₜ=ln M+k₁*t 2

Mₜ=M₀+k_{H}*t^{1/2} 3

In these equations, Mₜ is the cumulative amount of drug released at any specified time point and M₀ is the dose of active substance incorporated in the pharmaceutical composition. k₀, k₁ and k_{H} are rate constants for zero-order, first-order and Higuchi's equation, respectively.

One aspect of the invention, relates to a composition comprising the first component having a zero-order dissolution release profile. Another aspect relates to a first-order dissolution release profile. A further aspect relates to a square-root (Higuchi's equation) dissolution release profile.

In a further aspect of the invention, a controlled release pharmaceutical composition is used for delivery of the first component comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, a pharmaceutically acceptable polymer(s) and optionally pharmaceutically acceptable excipients or additives.

In one aspect of the invention, a controlled release pharmaceutical composition is used for delivery of the first component comprising as an active substance from 10% to 90% by weight of one or more fumaric acid esters selected from di-(C₁-C₅)alkylesters of fumaric acid and mono-(C₁-C₅)alkylesters of fumaric acid, or a pharmaceutically acceptable salt thereof, from 2% to 40% by weight pharmaceutically acceptable polymer(s), and from 1% to 40% by weight hydrophilic excipient(s), and optionally pharmaceutically acceptable excipients or additives.

In another aspect of the invention, a controlled release pharmaceutical composition is used for delivery of the first component comprising as an active substance from 40% to 60% by weight of one or more fumaric acid esters selected from di-(C₁-C₅)alkylesters of fumaric acid and mono-(C₁-C₅)alkylesters of fumaric acid, or a pharmaceutically acceptable salt thereof, from 15% to 25% by weight pharmaceutically acceptable polymer(s), and from 2% to 15% by weight hydrophilic excipient(s), and optionally pharmaceutically acceptable excipients or additives.

In a further aspect of the invention, a controlled release pharmaceutical composition is used for delivery of the first component comprising as an active substance from 65% to 80% by weight of one or more fumaric acid esters selected from di-(C₁-C₅)alkylesters of fumaric acid and mono-(C₁-C₅)alkylesters of fumaric acid, or a pharmaceutically acceptable salt thereof, from 10% to 25% by weight pharmaceutically acceptable polymer(s), and from 2% to 15% by weight hydrophilic excipient(s), and optionally pharmaceutically acceptable excipients or additives.

In a further aspect of the invention, a controlled release pharmaceutical composition is used for delivery of the first component comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives. In a further aspect, the amount of fumaric ester(s) is 42% to 48% by weight. In another aspect, the amount of pharmaceutically acceptable polymer(s) is 9% to 14% by weight. In a further aspect the invention relates to a pharmaceutical composition further comprising from 0.1% to 5% by weight hydrophilic excipient(s). In another aspect of the invention, the pharmaceutical composition comprises from 1% to 4% by weight hydrophilic excipient(s). In a further aspect of the invention, the pharmaceutical composition is in the form of a tablet. In a further aspect of the invention, the pharmaceutical composition is provided with an enteric coating.

In one aspect of the invention, the pharmaceutically acceptable polymer used in above controlled release formulation of the first component is a film-binding polymer. Examples of “pharmaceutically acceptable polymer(s)” comprises but are not limited to one or more selected from the group consisting of ethylcellulose (e.g. Ethocel® NF), methacrylic acid copolymer, and acrylic acid copolymer, such as ammonio methacrylate copolymer type A (e.g. Eudragit® RL30D) and/or B or methacrylic acid copolymer A and/or B. Other examples comprise but are not limited to polyvinyl acetate polymer (e.g. Kollicoat SR30D) and methacryl acetate polymer (e.g. Kollicoat MAE 30DP).

In one aspect of the invention, examples of “pharmaceutically acceptable polymer(s)” comprise but are not limited to one or more selected from the group consisting of ethylcellulose (e.g. Ethocel® NF), methacrylic acid copolymer such as methacrylic acid copolymer A and/or B, acrylic acid copolymer such as vinyl-pyrrolidone acrylic acid copolymer and ethyl acrylic acid copolymer, and ammonio methacrylate copolymer, such as ammonio methacrylate copolymer type A (e.g. Eudragit® RL30D) and/or B (e.g. Eudragit® RS30D). Other examples comprise but are not limited to polyvinyl acetate polymer (e.g. Kollicoat SR30D) and methacryl acetate polymer (e.g. Kollicoat MAE 30DP).

Examples of “hydrophilic excipient(s)” comprises but are not limited to polyethylene glycol (PEG), povidone, hydroxylpropyl cellulose (HPC), hydroxyethyl starch (HES) or hydroxypropyl methyl cellulose (HPMC) or a material with similar properties, or a combination thereof.

In a further aspect of the invention, the first component is delivered in a controlled release pharmaceutical composition, wherein the pharmaceutically acceptable polymer is ethyl cellulose.

In another aspect of the invention, the first component is delivered in a controlled release pharmaceutical composition, wherein the hydrophilic excipient is hydroxylpropyl cellulose.

In another aspect of the invention, the first component is delivered in a controlled release pharmaceutical composition, wherein the hydrophilic excipient is polyethylene glycol.

In yet another aspect of the invention, a controlled release pharmaceutical composition is used for delivery of the first component comprising as an active substance from 10% to 90% by weight of one or more fumaric acid esters selected from di-(C₁-C₅)alkylesters of fumaric acid and mono-(C₁-C₅)alkylesters of fumaric acid, or a pharmaceutically acceptable salt thereof, and 2% to 40% by weight methacrylic acid copolymer A and B in a weight ratio between 1:9 and 9:1, and Optionally pharmaceutically acceptable excipients or additives.

In a further aspect of the invention, a controlled release pharmaceutical composition is used for delivery of the first component comprising from 50% to 90% of one or more fumaric acid esters selected from di-(C₁-C₅)alkylesters of fumaric acid and mono-(C₁-C₅)alkylesters of fumaric acid, or a pharmaceutically acceptable salt thereof.

Various controlled release formulations, not limiting the scope of the present invention for delivery of the first component, are described hereafter (all concentrations based on the final tablet):

### Granules

In one aspect, granules may be prepared by mixing and/or granulating the active substance at a concentration of about 10 to about 90%, such as of about 20 to about 80%, of about 30 to about 75%, or of about 40 to about 70%, especially from about 50 to about 70%, with granulating excipients, such as pharmaceutical acceptable polymers, e.g. ethylcellulose such as Ethocel® NF premium, or methacrylic/acrylic acid copolymers, or ammonio methacrylate copolymers, such as ammonio methacrylate copolymer type A or B, or methacrylic acid copolymer A or B, or polyvinyl acetate polymer, or methacryl-ethylacetate polymer, incorporated at a concentration between about 8 to about 15%. Hydrophilic excipients such as polyethylene glycol (PEG), povidone, hydroxylpropyl cellulose (HPC), hydroxyethyl starch (HES) or hydroxypropyl methyl cellulose (HPMC) at a concentration of about 0.1 to about 5% and/or pharmaceutically acceptable surfactants with HLB values above 8 at a concentration of about 0.01 to about 3% may be incorporated.

### Tablets

Tablets may be based on granules. When it comes to producing tablets in large scale, especially on a rotary machine, further excipients to increase flow ability or to improve tabletting-behaviour may be needed. As filling and binding excipients, if required, mention can be made of e.g. microcrystalline cellulose, such as Avicel® 102, and cellulose at a concentration of about 1 to about 60%, crystalline, spray dried or granulated lactose monohydrate e.g. Tablettose®, as well as anhydrous lactose, at a concentration of about 5 to about 60%, sugar alcohols, such as sorbitol and mannitol, at a concentration of about 0 to about 40% and modified starch at a concentration of about 0 to about 40%. Furthermore disintegration agents such as starch and starch-derivates such as sodium starch glycolate (at a concentration of about 0.2 to about 10%), crospovidone (at a concentration of about 0.2 to about 10%), sodium carboxymethylcellulose (at a concentration of about 0.1 to about 10%), glidants such as colloidal anhydrous and hydrous silica (at a concentration of about 0.2 to about 4%), and lubricants, e.g. magnesium stearate, calcium behenate, and calciumarachinate (at a concentration of about 0.2 to about 3%) or sodium stearyl fumarate (at a concentration of about 1 to about 8%) can be added. In one aspect tablets may be based on a mixture of granules comprising active substance and placebo granules without active substance.

As mentioned above, a suitable controlled release providing the first component has in one aspect a pH controlled release (also known as a pH dependent release) of the fumaric acid ester. Normally, the release is designed so that only a small amount, if any, of the fumaric acid ester is released in the stomach (pH up to about 3), whereas the remainder i.e. the majority of fumaric acid ester is released in the intestines (pH shifts to about 6-7). Such a pH controlled release can be obtained by providing a composition of the invention with an enteric coating (the whole composition or, if the composition is a multiparticulate composition, the individual units).

### Dosage of First Component

The first component can be provided in dosages having different content of fumaric acid present, such as kits containing two or more containers e.g. with compositions having various amounts of the fumaric acid included. Such kits are suitable for use in those situations where an increasing dosage is required over time. A normal up-scale of the dosage is given below:

| Week | Morning | Noon | Evening | Strength |
|---|---|---|---|---|
| 1 | 1 | — | — | A |
| 2 | 1 | — | 1 | A |
| 3 | 1 | — | 1 | B |
| 4 | 1 | — | — | B |
| 5 | 1 | — | 1 | B |
| 6 | 1 | 1 | 1 | B |
| 7 | 2 | 1 | 1 | B |
| 8 | 2 | 1 | 2 | B |
| 9 | 2 | 2 | 2 | B |

A corresponds to a low strength such as about 30 mg dimethylfumarate (or a corresponding effective dose of another fumaric acid ester)
B corresponds to a higher strength such as about 120 mg dimethylfumarate (or a corresponding effective dose of another fumaric acid ester)

In one aspect of the invention, a controlled release pharmaceutical composition is provided wherein the amount of one or more fumaric acid esters selected from di-(C₁-C₅)alkylesters of fumaric acid and mono-(C₁-C₅)alkylesters of fumaric acid, or a pharmaceutically acceptable salt thereof, in a dosage form is from 50 mg to 1000 mg active substance, such as 90 mg to 600 mg active substance.

In one aspect of the invention, the first component is provided in a controlled release pharmaceutical composition, wherein the amount of one or more fumaric acid esters selected from di-(C₁-C₅)alkylesters of fumaric acid and mono-(C₁-C₅)alkylesters of fumaric acid, or a pharmaceutically acceptable salt thereof, in a dosage form is from 90 mg to 500 mg active substance, such as 90, 120, 180, 240, 360, 450, 480 or 500 mg active substance. In a further aspect of the invention, the amount of the first component is 120, 180 or 240 mg active substance. In yet a further aspect of the invention, the amount of the first component is 180 or 360 mg active substance.

In yet a further aspect of the invention, the amount of the first component in a dosage form is 120 mg active substance. In yet a further aspect of the invention, the amount of active substance in a dosage form is 180 mg active substance. In yet a further aspect of the invention, the amount of active substance in a dosage form is 240 mg active substance. In yet a further aspect of the invention, the amount of active substance in a dosage form is 360 mg active substance. In yet a further aspect of the invention, the amount of active substance in a dosage form is 450 mg active substance. In yet a further aspect of the invention, the amount of active substance in a dosage form is 480 mg active substance. In yet a further aspect of the invention, the amount of active substance in a dosage form is 500 mg active substance.

In yet a further aspect of the invention, of the first component in a dosage form is 50 mg active substance. In yet a further aspect of the invention, the amount of active substance in a dosage form is 100 mg active substance. In yet a further aspect of the invention, the amount of active substance in a dosage form is 150 mg active substance. In yet a further aspect of the invention, the amount of active substance in a dosage form is 200 mg active substance. In yet a further aspect of the invention, the amount of active substance in a dosage form is 250 mg active substance. In yet a further aspect of the invention, the amount of active substance in a dosage form is 300 mg active substance. In yet a further aspect of the invention, the amount of active substance in a dosage form is 350 mg active substance. In yet a further aspect of the invention, the amount of active substance in a dosage form is 400 mg active substance. In yet a further aspect of the invention, the amount of active substance in a dosage form is 450 mg active substance. In yet a further aspect of the invention, the amount of active substance in a dosage form is 600 mg active substance. In yet a further aspect of the invention, the amount of active substance in a dosage form is 700 mg active substance. In yet a further aspect of the invention, the amount of active substance in a dosage form is 800 mg active substance. In yet a further aspect of the invention, the amount of active substance in a dosage form is 900 mg active substance. In yet a further aspect of the invention, the amount of active substance in a dosage form is 1000 mg active substance.

The daily dosage of the controlled release pharmaceutical composition that is administered to treat a patient depends on a number of factors among which are included, without limitation, weight and age and the underlying causes of the condition or disease to be treated, and is within the skill of a physician to determine. The daily dosage can be e.g. from 240 to 360 mg active substance given in one to three doses, in another aspect from 360 to 480 mg active substance given in one to three doses, in another aspect 480 to 600 mg active substance given in one to three doses, in another aspect 600 to 720 mg active substance given in one to three doses, in another aspect 720 to 840 mg active substance given in one to three doses, in another aspect 840 to 960 mg active substance given in one to three doses and in yet another aspect 960 to 1080 mg active substance given in one to three doses, and in yet another aspect 700 to 1500 mg active substance given in one to three doses.

In one aspect of the invention, the controlled release pharmaceutical composition providing the first component is in the form of a capsule.

In another aspect of the invention, the controlled release pharmaceutical composition in the form of a tablet is provided, such as a tablet which has a shape that makes it easy and convenient for a patient to swallow e.g. a tablet which has a rounded or a rod-like shape without any sharp edges.

In one aspect, the invention relates to adjunctive therapy with the following combinations of a first and second component:

As a first component: BG-12/Panaclar™, Fumaderm, Fumaraat®, or a controlled release formulation comprising dimethyl fumarate as active substance.
As a second component: a DP selective compound such as MK-0524, Paracetamol, Naproxen, Salicylic acid, ibuprofen, Meloxicam, piroxicam, Dexibuprofen, Salsalate, or Diclofenac.

In another aspect, the invention relates to adjunctive therapy with the following combinations of a first and second component:

As a first component: BG-12/Panaclar™, Fumaderm, Fumaraat®, or a controlled release formulation comprising dimethyl fumarate as active substance.
As a second component: a DP selective compound such as MK-0524, Naproxen, Salicylic acid, ibuprofen, Meloxicam, piroxicam, Dexibuprofen, Salsalate, or Diclofenac.

In yet another aspect, the invention relates to adjunctive therapy with the following combinations of a first and second component:

As a first component: BG-12/Panaclar™, Fumaderm, Fumaraat®, or a controlled release formulation comprising dimethyl fumarate as active substance.
As a second component: a DP selective compound such as MK-0524, Naproxen, ibuprofen, Meloxicam, piroxicam, Dexibuprofen, Salsalate, or Diclofenac.

### Administration

In one aspect, the invention relates to a pharmaceutical kit for administering the first component and the second component having reduced capacity to provoke a flushing reaction in a subject, the kit is including a plurality of doses, at least one dose including a pharmaceutical composition comprising a first component, and a flush-reducing amount and dosage form of a second component. The dose can include a sustained release, or immediate release form or a combination thereof, and optionally, a delayed release form.

In one embodiment, the kit includes a predose for administering to the subject a flush-reducing regimen of the second component prior to beginning administration of the first component. The predose can include a sustained release, delayed release or immediate release form or a combination of two or more thereof.

In one aspect of the invention, the second component is administered up to 12 hours before the first component, such as up to about 11 hours, 10 hours, 9 hours, 8 hours, 7 hours, 6 hours, 5 hours, 4 hours, 3 hours, 2 hours, 1 hours, 30 min., 15 min., or min. before the first component. In another aspect, the second component is administered up to 6 hours after the first component, such as up to about 5 hours, 4 hours, 3 hours, 2 hours, 1 hours, 30 min., 15 min., or 5 min. after the first component. In yet a further aspect, the first and the second component is administered at the same time.

The components of the present invention may be administered together with a meal or in relation to a meal such as e.g. in a time period corresponding to a range from at least about 30 minutes before a meal to about 2 hours after the meal, or the composition may be administered at any specific point(s) in time during the day.

In one embodiment, the total daily dose is given at bedtime, such as up to or about 30 minutes before bedtime, up to or about 60 minutes before bedtime, up to or about 90 minutes before bedtime, up to or about 120 minutes before bedtime or up to or about 180 minutes before bedtime.

It will be understood that while the use of a single first component compound is preferred, combinations of two or more first component compounds may be used as a first component if necessary or desired. Similarly, while the use of a single second component compound is preferred, combinations of two or more second components may be used as a second component if necessary or desired.

It will also be understood that while the use of a single drug as a first component compound is preferred, combinations of two or more drugs may be used as a first component if necessary or desired. Similarly, while the use of a single drug as a second component compound is preferred, combinations of two or more drugs may be used as a second component if necessary or desired.

The invention relates in one aspect to a pharmaceutical composition which comprises a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing.

The invention relates in a further aspect to a pharmaceutical composition which comprises a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing selected from the group consisting of:
a. a prostaglandin modulator,
b. a dietary fiber,
c. a composition comprising inhibitors of mast cell activation and secretion of inflammatory biochemicals,
d. a mineral salt, and
e. a TNF alpha inhibitor.

The adjunctive therapy of the present invention is contemplated to be suitable for use in the treatment of one or more of the following conditions:
a. Psoriasis
b. Psoriatic arthritis
c. Neurodermatitis
d. Inflammatory bowel disease, such as
   i. Crohn's disease
   ii. Ulcerative colitis
e. Polyarthritis
f. Multiple sclerosis (MS)
g. Juvenile-onset diabetes mellitus
h. Hashimoto's thyroiditis
i. Grave's disease
j. SLE (systemic lupus erythematosus)
k. Sjögren's syndrome
l. Pernicious anemia
m. Chronic active (lupoid) hepatitis
n. Rheumatoid arthritis (RA)
o. Optic neuritis

Moreover, the adjunctive therapy of the present invention may be used in the treatment of
1. Pain such as radicular pain, pain associated with radiculopathy, neuropathic pain or sciatica/sciatic pain
2. Organ transplantation (prevention of rejection)
3. Sarcoidosis
4. Necrobiosis lipoidica
5. Granuloma annulare

Moreover, the adjunctive therapy of the present invention may be used in the treatment of lupus nephritis, myasthenia gravis, uveitis, refractory uveitis, vernal conjunctivitis, pemphigus vulgaris, or scleroderma.

The invention relates in one aspect to the use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt;
   for the manufacture of a medicament for treating an autoimmune and/or inflammatory disorder.

The invention relates in a further aspect to the use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and a second component which is a substance that reduces or eliminates flushing, such as selected from the group consisting of:
a. a prostaglandin modulator,
b. a dietary fiber,
c. a composition comprising inhibitors of mast cell activation and secretion of inflammatory biochemicals,
d. a mineral salt, and
e. a TNF alpha inhibitor
   for the manufacture of a medicament for treating an autoimmune and/or inflammatory disorder.

The invention relates in one aspect to an adjunctive treatment of an autoimmune and/or inflammatory disorder which is psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.

In a further aspect of the invention, the autoimmune and/or inflammatory disorder is psoriasis.

In a further aspect of the invention, the autoimmune and/or inflammatory disorder is multiple sclerosis.

Psoriasis has been proposed to potentially be associated with Crohn's disease (Najarian D J, Gottlieb A B, Connections between psoriasis and Crohn's disease. J Am Acad Dermatol. 2003 June; 48(6):805-21), celiac disease (Ojetti V et al, High prevalence of celiac disease in psoriasis. Am J Gastroenterol. 2003 November; 98(11):2574-5.), psychiatric or psychological disease, such as depression or a life crisis (Gupta M A, Gupta A K, Psychiatric and psychological co-morbidity in patients with dermatologic disorders: epidemiology and management. Am J Clin Dermatol. 2003; 4(12):833-42. and Mallbris L et al, Psoriasis phenotype at disease onset: clinical characterization of 400 adult cases. J Invest Dermatol. 2005 March; 124(3):499-504.), overweight, diabetes mellitus, excess consumption of alcohol/alcoholism, as well as psoriatic arthritis.

The adjunctive therapy of the present invention is carried out by administering a first component together with the second component in any manner which provides effective levels of the compounds in the body at the same time. Oral administration of the adjunctive combination is preferred. They may be administered together, in a single dosage form, or they may be administered separately in any sequential order or concomitantly.

However, oral administration is not the only route or even the only preferred route. For example, transdermal administration may be very desirable for patients who are forgetful or petulant about taking oral medicine. One of the drugs may be administered by one route, such as oral, and the others may be administered by the transdermal, percutaneous, intravenous, intramuscular, intranasal or intrarectal route, in particular circumstances. The route of administration may be varied in any way, limited by the physical properties of the drugs and the convenience of the patient and the caregiver.

The adjunctive combination may be administered as a single pharmaceutical composition, and so pharmaceutical compositions incorporating both compounds are important embodiments of the present invention. Such compositions may take any physical form which is pharmaceutically acceptable, but orally usable pharmaceutical compositions are particular embodiments. Such adjunctive pharmaceutical compositions contain an effective amount of each of the compounds, which effective amount is related to the daily dose of the compounds to be administered. Each dosage unit may contain the daily doses of both components, or may contain a fraction of the daily doses, such as one third of the doses. Alternatively, each dosage unit may contain the entire dose of one of the component compounds, and a fraction of the dose of the other component compound.

In such case, the patient would daily take one of the combination dosage units, and one or more units containing only the other compounds. The amounts of each drug to be contained in each dosage unit depends on the identity of the drugs chosen for the therapy, and other factors such as the indication for which the adjunctive therapy is being given.

The inert ingredients and manner of formulation of the adjunctive pharmaceutical compositions are conventional, except for the presence of the combination of the present invention. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The activity of the adjunctive combinations does not depend on the nature of the composition, so the compositions are chosen and formulated solely for convenience and economy. Any of the combinations may be formulated in any desired form of composition. Some discussion of different compositions will be provided, followed by some typical formulations.

Capsules are prepared by mixing the compound with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like.

Polyethylene glycol, ethylcellulose and waxes can also serve as binders.

A lubricant is necessary in a tablet formulation to prevent the tablet and punches from sticking in the die. The lubricant is chosen from such slippery solids as talc, magnesium and calcium stearate, stearic acid and hydrogenated vegetable oils. Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate.

Tablets are often coated with sugar as a flavor and sealant. The compounds may also be formulated as chewable tablets, by using large amounts of pleasant-tasting substances such as mannitol in the formulation, as is now well-established practice.

Tablet may be designed to be divided into two or more parts.

Instantly dissolving tablet-like formulations are also now frequently used to assure that the patient consumes the dosage form, and to avoid the difficulty in swallowing solid objects that bothers some patients. Capsules and tablets are the preferred dosage unit form.

When it is desired to administer the combination as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches typically comprise a resinous composition in which the drugs will dissolve, or partially dissolve, which is held in contact with the skin by a film which protects the composition. Other, more complicated patch compositions are also in use, particularly those having a membrane pierced with innumerable pores through which the drugs are pumped by osmotic action.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are described. All publications mentioned herein are incorporated herein by reference to disclose and describe the methods and/or materials in connection with which the publications are cited. It must be noted that as used herein and in the appended claims, the singular forms “a”, “an”, and “the” include plural referents unless the context clearly dictates otherwise. The patents and publications discussed herein are provided solely for their disclosure prior to the filing date of the present application. Nothing herein is to be construed as an admission that the present invention is not entitled to antedate such patent or publication by virtue of prior invention. Further, the dates of publication provided may be different from the actual publication dates which may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of treating an autoimmune or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and in a therapeutically effective amount a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt.

2. A method of treating an autoimmune or inflammatory disorder in a human patient in need of such treatment comprising administering to said patient in a therapeutically effective amount a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and in a therapeutically effective amount a second component which is a substance that reduces or eliminates flushing.

3. The method according to any one of the claims 1-2, wherein said disorder is both an autoimmune and inflammatory disorder.

4. The method according to any one of the claims 2-3, wherein the second component is selected from the group consisting of:
a. a prostaglandin modulator,
b. a dietary fiber,
c. a composition comprising inhibitors of mast cell activation and secretion of inflammatory biochemicals,
d. a mineral salt, and
e. a TNF alpha inhibitor.

5. The method according to any one of the claims 1-4, wherein the first component is a mono-(C₁-C₅)alkylester of fumaric acid that is present in the form of a pharmaceutically acceptable salt.

6. The method according to any one of the claims 1-5, wherein the first component is a metal salt such as a salt selected from the group consisting of alkali metal salts and alkaline earth metal salts.

7. The method according to claim 6, wherein the first component is selected from the group consisting of a sodium, potassium, calcium, magnesium and zinc salt.

8. The method according to any one of the claims 1-7, wherein the first component is dimethylfumarate.

9. The method according to any one of the claims 1-7, wherein the first component is monomethylfumarate.

10. The method according to claim 9, wherein the first component is monomethylfumarate present in the form of a salt selected from the group consisting of sodium, potassium, calcium, magnesium and zinc salt.

11. The method according to any one of the claims 1-4, wherein the first component is provided in the form of Fumaderm®.

12. The method according to any one of the claims 1-11, wherein the second component is a prostaglandin modulator.

13. The method according to claim 12, wherein the second component is a prostaglandin modulator selected from the group consisting of NSAIDs, corticosteroids and prostaglandin inhibitors

14. The method according to claim 13, wherein the second component is a prostaglandin inhibitor.

15. The method according to claim 14, wherein the second component is a prostaglandin D2 inhibitor.

16. The method according to claim 15, wherein the second component is a DP receptor antagonist.

17. The method according to claim 16, wherein the DP receptor antagonist selectively modulates the DP receptor and does not substantially modulate the CRTH2 receptor.

18. The method according to claim 17, wherein the DP receptor antagonist is

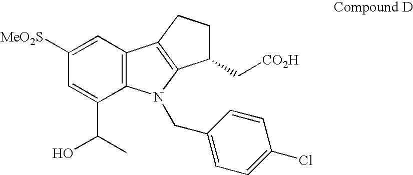

19. The method according to claim 17, wherein the DP receptor antagonist is MK-0524.

20. The method according to claim 13, wherein the second component is a NSAID.

21. The method according to claim 20, wherein the second component is a NSAID selected from the group consisting of meloxicam, piroxicam, naproxen, acetaminophen (paracetamol), ibuprofen, dexibuprofen, diclofenac, and salicylic acid.

22. The method according to claim 21, wherein the second component is a COX-2 inhibitor selected from the group consisting of rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6-dimethyl-6H-dibenzo[b,d]pyran carboxylic acid (CT-3), 2(5H)-Furanone, 5,5-dimethyl (1-methylethoxy) [4(methylsulfonyl)phenyl]-(DFP); Carprofen (RIMADYL), (Acetyloxy)-benzoic acid, 3-[(nitrooxy)methyl]phenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenoI (S-2474), 5(R)-Thio sulfonamide-3(2H)-benzofuranone (SVT-2016) and N-[3-(Formyl-amino) oxo phenoxy-4H benzopyran yl]methanesulfonamide (“T-614”); or a pharmaceutically acceptable salt thereof.

23. The method according to any one of the claims 1-11, wherein the second component is a dietary fiber.

24. The method according to claim 23, wherein the second component is guar gum.

25. The method according to any one of the claims 2-11, wherein the second component is a TNF alpha inhibitor.

26. The method according to claim 25, wherein the second component is an oral TNF alpha inhibitor.

27. The method according to claim 26, wherein the second component is an oral TNF alpha inhibitor selected from the group consisting of lenalidomide, ITF-2357, POL-647, thalidomide (Andrulis), thalidomide (Celgene), Pharmaprojects No. 6181, antisense compounds (Elan), antisense compounds (Isis), delmitide acetate, AGIX-4207, TNF-alpha antagonists (Dynavax), Genz-29155, CDC-394, NPI-1302a-3, ENMD-0995, ENMD-0997, PLR-14, UR-1505, TNF-alpha inhibitors (Morphochem), LMP-420, LMP-160, STA-6292, ISIS-104838, ABX/0402, CC-11006, IMiDs (class III, Celgene), CC-10004, CC-11050, PG-8395 and BKT-104.

28. The method according to any one of the claims 1-11, wherein the second component is a composition comprising inhibitors of mast cell activation and secretion of inflammatory biochemicals.

29. The method according to claim 28, wherein the second component is sodium chromoglycate.

30. The method according to any one of the claims 1-11, wherein the second component is a mineral salt.

31. The method according to claim 30, wherein the second component is a magnesium salt.

32. The method according to claim 30, wherein the second component is selected from the group consisting of calcium carbonate, magnesium carbonate and potassium carbonate.

33. The method according to any one of the claims 1-32, wherein the disorder is psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.

34. The method according to any one of the claims 1-32, wherein the disorder is psoriasis.

35. The method according to any one of the claims 1-32, wherein the disorder is multiple sclerosis.

36. The use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing selected from the group consisting of:
a. a prostaglandin modulator,
b. a dietary fiber
c. a composition comprising inhibitors of mast cell activation and secretion of inflammatory biochemicals, and
d. a mineral salt;
for the manufacture of a medicament for treating an autoimmune or inflammatory disorder.

37. The use of a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof in a controlled release composition, and a second component which is a substance that reduces or eliminates flushing for the manufacture of a medicament for treating an autoimmune or inflammatory disorder.

38. The method according to any one of the claims 36-37, wherein said disorder is both an autoimmune and inflammatory disorder.

39. The use according to any one of the claims 37-38, wherein the second component is selected from the group consisting of:
a. a prostaglandin modulator,
b. a dietary fiber,
c. a composition comprising inhibitors of mast cell activation and secretion of inflammatory biochemicals,
d. a mineral salt, and
e. a TNF alpha inhibitor.

40. A pharmaceutical composition which comprises a first component which is one or more fumaric acid esters selected from the group consisting of di-(C₁-C₅)alkylesters of fumaric acid and mono-(C₁-C₅)alkylesters of fumaric acid, or a pharmaceutically acceptable salt thereof, and a second component which is a substance that reduces or eliminates flushing.

41. The pharmaceutical composition according to claim 40, wherein the second component is selected from the group consisting of:
a. a prostaglandin modulator,
b. a dietary fiber,
c. a composition comprising inhibitors of mast cell activation and secretion of inflammatory biochemicals,
d. a mineral salt, and
e. a TNF alpha inhibitor.

42. The pharmaceutical composition according to any one of the claims 40-42, wherein the first component is a mono-(C₁-C₅)alkylester of fumaric acid that is present in the form of a pharmaceutically acceptable salt.

43. The pharmaceutical composition according to any one of the claims 40-42, wherein the first component is a metal salt such as a salt selected from the group consisting of alkali metal salts and alkaline earth metal salts.

44. The pharmaceutical composition according to claim 43, wherein the first component is selected from the group consisting of a sodium, potassium, calcium, magnesium and zinc salt.

45. The pharmaceutical composition according to any one of the claims 40-41, wherein the first component is dimethylfumarate.

46. The pharmaceutical composition according to any one of the claims 40-41, wherein the first component is monomethylfumarate.

47. The pharmaceutical composition according claim 46, wherein the first component is monomethylfumarate present in the form of a salt selected from the group consisting of its sodium, potassium, calcium, magnesium and zinc salt.

48. The pharmaceutical composition according to any one of the claims 40-41, wherein the second component is a prostaglandin modulator.

49. The pharmaceutical composition according claim 48, wherein the second component is a prostaglandin modulator selected from the group consisting of NSAIDs, corticosteroids and prostaglandin inhibitors.

50. The pharmaceutical composition according claim 49, wherein the second component is a prostaglandin inhibitor.

51. The pharmaceutical composition according claim 50, wherein the second component is a prostaglandin D2 inhibitor.

52. The pharmaceutical composition according claim 51, wherein the second component is a DP receptor antagonist.

53. The pharmaceutical composition according claim 52, wherein the DP receptor antagonist selectively modulates the DP receptor and does not substantially modulate the CRTH2 receptor.

54. The pharmaceutical composition according claim 53, wherein the DP receptor antagonist is

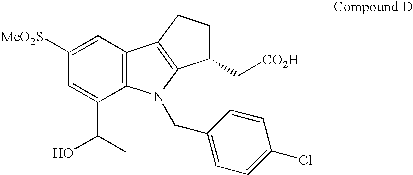

55. The pharmaceutical composition according claim 53, wherein the DP receptor antagonist is MK-0524.

56. The pharmaceutical composition according claim 49, wherein the second component is a NSAID.

57. The pharmaceutical composition according claim 56, wherein the second component is a NSAID selected from the group consisting of meloxicam, piroxicam, naproxen, acetaminophen (paracetamol), ibuprofen, dexibuprofen, diclofenac, and salicylic acid.

58. The pharmaceutical composition according claim 57, wherein the second component is a COX-2 inhibitor selected from the group consisting of rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6-dimethyl-6H-dibenzo[b,d]pyran carboxylic acid (CT-3), 2(5H)-Furanone, 5,5-dimethyl (1-methylethoxy) [4(methylsulfonyl)phenyl]-(DFP); Carprofen (RIMADYL), (Acetyloxy)-benzoic acid, 3-[(nitrooxy)methyl]phenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenoI (S-2474), 5(R)-Thio sulfonamide-3(2H)-benzofuranone (SVT-2016) and N-[3-(Formyl-amino) oxo phenoxy-4H benzopyran yl]methanesulfonamide (“T-614”); or a pharmaceutically acceptable salt thereof.

59. The pharmaceutical composition according to any one of the claims 40-41, wherein the second component is a dietary fiber.

60. The pharmaceutical composition according claim 59, wherein the second component is guar gum.

61. The pharmaceutical composition according to any one of the claims 40-41, wherein the second component is a TNF alpha inhibitor.

62. The pharmaceutical composition according claim 61, wherein the second component is an oral TNF alpha inhibitor.

63. The pharmaceutical composition according claim 62, wherein the second component is an oral TNF alpha inhibitor selected from the group consisting of lenalidomide, ITF-2357, POL-647, thalidomide (Andrulis), thalidomide (Celgene), Pharmaprojects No. 6181, antisense compounds (Elan), antisense compounds (Isis), delmitide acetate, AGIX-4207, TNF-alpha antagonists (Dynavax), Genz-29155, CDC-394, NPI-1302a-3, ENMD-0995, ENMD-0997, PLR-14, UR-1505, TNF-alpha inhibitors (Morphochem), LMP-420, LMP-160, STA-6292, ISIS-104838, ABX/0402, CC-11006, IMiDs (class III, Celgene), CC-10004, CC-11050, PG-8395 and BKT-104.

64. The pharmaceutical composition according to any one of the claims 40-41, wherein the second component is a composition comprising inhibitors of mast cell activation and secretion of inflammatory biochemicals.

65. The pharmaceutical composition according claim 64, wherein the second component is sodium chromoglycate.

66. The pharmaceutical composition according to any one of the claims 40-41, wherein the second component is a mineral salt.

67. The pharmaceutical composition according claim 66, wherein the second component is a magnesium salt.

68. The pharmaceutical composition according claim 67, wherein the second component is selected from the group consisting of calcium carbonate, magnesium carbonate and potassium carbonate.

69. A method of treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare which method comprises administering to a patient in need thereof, an effective dosage of a pharmaceutical composition according to any one of claims 40-68.

70. The method according to claim 67, wherein the disorder is psoriasis.

71. The method according to claim 67, wherein the disorder is multiple sclerosis.
